SEQUENCE LISTING (6 sequences)

SEQ ID NO: 1 - RNA, 20 nt, synthetic construct
  source 1..20: mol_type = other RNA; note = guide RNA
ggcggctttagagcaacccg

SEQ ID NO: 2 - RNA, 20 nt, synthetic construct
  source 1..20: mol_type = other RNA; note = guide RNA
cgtggagaaggagtatcggt

SEQ ID NO: 3 - RNA, 20 nt, synthetic construct
  source 1..20: mol_type = other RNA; note = guide RNA
cgagatgtggacacctacta

SEQ ID NO: 4 - DNA, 12 bp, synthetic construct
  source 1..12: mol_type = other DNA; note = oligomer
aggaattaagga

SEQ ID NO: 5 - DNA, 12 bp, synthetic construct
  source 1..12: mol_type = other DNA
  modified_base 7: mod_base = OTHER; note = n is EdU 5-ETHYNYL-2-DEOXYURIDINE
aggaatnaagga

SEQ ID NO: 6 - DNA, 12 bp, synthetic construct
  source 1..12: mol_type = other DNA
  modified_base 6: mod_base = OTHER; note = n is EdU 5-ETHYNYL-2-DEOXYURIDINE
  modified_base 7: mod_base = OTHER; note = n is EdU 5-ETHYNYL-2-DEOXYURIDINE
aggaannaagga

(19) United States
(12) Patent Application Publication
(10) Pub. No.: US 2026/0034158 A1
(43) Pub. Date: Feb. 5, 2026

(54) METHODS OF TREATING CANCER OF THE CENTRAL NERVOUS SYSTEM COMPRISING 5-ETHYNYL-2'-DEOXYURIDINE

(71) Applicant: The University of North Carolina at Chapel Hill, Chapel Hill, NC (US)

(72) Inventors: Aziz Sancar, Chapel Hill, NC (US); Li Wang, Chapel Hill, NC (US)

(21) Appl. No.: 18/996,070

(22) PCT Filed: Jul. 20, 2023
(86) PCT No.: PCT/US2023/070594

§ 371 Date: Jan. 17, 2025

Related U.S. Application Data
(60) Provisional application No. 63/390,720, filed on Jul. 20, 2022.

(51) Int. Cl. A61K 31/7072; A61K 31/131; A61K 31/137; A61K 31/138; A61K 31/198; A61K 31/277; A61K 31/519; A61K 31/675; A61K 31/704; A61K 31/7068; A61K 33/243

(52) U.S. Cl. CPC A61K 31/7072; A61K 31/131; A61K 31/137; A61K 31/138; A61K 31/198; A61K 31/277; A61K 31/519; A61K 31/675; A61K 31/704; A61K 31/7068; A61K 33/243

(57) ABSTRACT

The present invention relates generally to the fields of cancer cell biology, cancer therapeutics for cancers located within in the central nervous system, thymidine analogs and cellular nucleotide excision repair mechanisms. More specifically, the invention relates to the use of EdU in methods of treating cancers located within in the central nervous system, and methods of inhibiting and/or reducing growth of a cancer or cancer cell.

### STATEMENT OF PRIORITY

This application claims the benefit, under 35 U.S.C. § 119(e), of U.S. Provisional Application No. 63/390,720, filed Jul. 20, 2022, the entire contents of which is incorporated by reference herein in its entirety.

### STATEMENT OF GOVERNMENT SUPPORT

This invention was made with government support under Grant Numbers GM118102 and ES02755 awarded by the National Institutes of Health. The government has certain rights in the invention.

### STATEMENT REGARDING ELECTRONIC FILING OF A SEQUENCE LISTING

A Sequence Listing in XML format, entitled 5470-958WO_ST26.xml, 8,607 bytes in size, generated on Jul. 20, 2023, and filed herewith, is hereby incorporated by reference in its entirety for its disclosures.

### FIELD OF THE INVENTION

The present invention relates generally to the fields of cancer cell biology, cancer therapeutics for cancers located within in the central nervous system, thymidine analogs and cellular nucleotide excision repair. More specifically, the invention relates to the use of 5-ethynyl-2′-deoxyuridine (EdU) in methods of treating cancers located within in the central nervous system, and methods of inhibiting and/or reducing growth of a cancer or cancer cell.

### BACKGROUND OF THE INVENTION

Cancers and tumors in the brain and spinal cord are called central nervous system (CNS) tumors. A tumor may be benign, meaning it does not have cancer cells, or malignant, meaning cancer is present. A tumor that starts in another part of the body and spreads to the brain or the spinal cord is called a metastatic CNS tumor. The brain and spine are two common sites of metastases.

The blood-brain barrier (BBB) and blood-spinal cord barrier (BSCB) are highly selective semipermeable anatomical interfaces that prevent substances in the circulating blood from non-selectively crossing into the extracellular fluid (the cerebrospinal fluid or CSF) of the CNS or the spinal cord, thereby protecting the neural brain and spinal tissue. While the BBB and BSCB act effectively to protect brain tissue from circulating pathogens and other toxic substances, many cancer therapeutics and antibodies also cannot cross the barrier. Effective therapeutic options for cancers of the central nervous system are thus limited. Further, in some cases, a therapeutic agent has to be administered directly into the brain or cerebrospinal fluid, which presents additional limitations on treatment options.

The present invention overcomes previous shortcomings in the art by providing methods and compositions for treating cancers located within in the central nervous system.

### SUMMARY OF THE INVENTION

One aspect of the invention provides a method of treating a cancer located within the central nervous system in a subject in need thereof, comprising administering to the subject a therapeutically effective amount of EdU or a nucleic acid molecule or composition comprising the same, thereby treating the cancer within the central nervous system of the subject.

Another aspect of the invention provides a method of inhibiting and/or reducing growth of a cancer located within the central nervous system in a subject in need thereof, comprising administering to the subject a therapeutically effective amount of EdU or a nucleic acid molecule or composition comprising the same, thereby inhibiting and/or reducing growth of the cancer in the subject.

Another aspect of the invention provides a method of killing a cancer cell of a central nervous system cancer in a subject in need thereof, comprising: administering to the subject a therapeutically effective amount of EdU or a nucleic acid molecule or composition comprising the same, wherein the EdU contacts the cancer cell within the central nervous system of the subject, thereby killing the cancer cell of the central nervous cancer in the subject.

Another aspect of the invention provides a method of inhibiting and/or reducing proliferation of a cancer cell located within the central nervous system in a subject in need thereof, comprising: administering to the subject a therapeutically effective amount of EdU or a nucleic acid molecule or composition comprising the same, wherein the EdU contacts the cancer cell within the central nervous system of the subject, thereby inhibiting and/or reducing proliferation of the cancer cell in the subject.

In some embodiments, the cancer may be a spinal cancer or a brain cancer such as glioblastoma multiforme, oligodendroglioma, ependymoma, mixed glioma, choroid plexus tumors, ganglion cell tumors, embryonal tumors, meningioma, astrocytoma, lymphoma, pituitary tumors, craniopharyngioma, germ cell tumors, non-meningothelial mesenchymal tumors, pineal region tumors, medulloblastomas, cancerous cysts, and/or metastatic tumors.

Also provided are nucleic acid molecules, compositions and kits comprising EdU, for example, for the use of treating a cancer located in the central nervous system and/or any one of the methods disclosed herein.

### BRIEF DESCRIPTION OF THE DRAWINGS

FIG. 1 shows schematics of chemical structures and images of blots related to studies on the incorporation of thymidine analogs into the human genome and removal of EdU by nucleotide excision repair. (FIG. 1 panel A) Chemical structure of thymidine analogs used in this study. (FIG. 1 panel B) Slot blot assay to test the reactivity of thymidine analogs with anti-BrdU antibody. HeLa cells were cultured either in regular medium or medium containing 10 μM thymidine analog (EdU, BrdU, CldU, IdU, F-ara-EdU, or AmdU) for 24 h. Genomic DNA was isolated, loaded onto a nitrocellulose membrane, and incubated with anti-BrdU or anti-ssDNA antibody. Anti-BrdU antibody recognizes BrdU, EdU, CldU, and IdU labeled genomic DNA. (FIG. 1 panel C) Excision assay with anti-BrdU antibody immunoprecipitation. HeLa cells were treated with 10 μM thymidine analogs: EdU, BrdU, IdU, or CIdU for 24 h, then low-molecular-weight DNA was extracted by the Hirt procedure and immunoprecipitated with anti-BrdU antibodies. Purified, excised oligonucleotides were radiolabeled at the 3′ end together with a 50-mer as a spike-in internal control and separated on a DNA-sequencing gel along with a DNA ladder. (FIG. 1 panel D) Removal of EdU requires excision repair. Human NHF1 wild-type and XPA^{-/-}XPC^{-/-}CSB^{-/-} mutant cells (knockout [KO]) were treated with or without 10 μM EdU for 24 h. Then, cells were lysed by the Hirt procedure, and low-molecular-weight DNA in the supernatant was immunoprecipitated with anti-BrdU antibodies. The oligonucleotides were mixed with a 50-mer internal control, 3′ end-labeled, and analyzed on a DNA-sequencing gel. EdU excision product is produced by the wild-type NHF1 cells but not by the excision repair defective XPA^{-/-}XPC^{-/-}CSB^{-/-} cell line. (FIG. 1 panel E) In vivo excision assay with TFIIH antibody immunoprecipitation. HeLa cells were treated as in FIG. 1 panel C, and the primary excision products were isolated by TFIIH immunoprecipitation. Purified primary products were mixed with a 50-mer internal control oligonucleotide, 3′ end-radiolabeled, and separated on a DNA-sequencing gel along with a DNA ladder. The excised oligomers are 25 to 32 nt in length, as is the case in excision repair of bulky adducts. Low-molecular-weight excised oligomers are not observed in TFIIH IPs, because after the excision products are released from TFIIH, they are degraded, and as seen in FIG. 1 panels C and D, can be immunoprecipitated with anti-BrdU antibodies.

FIG. 2 shows a schematic and image of a blot related to studies showing EdU is a substrate for excision repair. Anti-BrdU antibody precipitates excision products from EdU-labeled, unirradiated cells. HeLa cells were either not treated with thymidine analog or treated with 10 μM EdU or BrdU for 24 h, then irradiated with UV (20 J/m²) where indicated. Red triangles indicate CPDs produced by UV exposure. After 1 h of repair in the presence of analog, excised oligonucleotides were separated from cells, immunoprecipitated with anti-BrdU antibodies, and then radiolabeled at the 3′ end using α-[³²P] ATP and TdT, and analyzed on a sequencing gel.

FIG. 3 shows a schematic and images of a blot and bar graphs related to studies showing EdU-substituted DNA is recognized as damage and excised by nucleotide excision repair in vitro. (FIG. 3 panel A) Schematic of substrate synthesis and in vitro excision of EdU by mammalian CFE. 140bp duplex substrates were synthesized with a ³²P label adjacent to a uniquely located modification. Modifications (red triangles) included a (6, 4) PP, an EdU, or two EdUs. Substrates were synthesized by phosphorylating, annealing, and ligating six component oligonucleotides. Ligation sites are indicated by dots. The central oligonucleotide containing the modification was labeled at the 5′ end with 32P (lighter dot site). A control UM substrate was also synthesized (not shown). Substrates were incubated with CFE, and unreacted full-length substrate and excised products were purified and resolved with a sequencing gel. (FIG. 3 panel B) Result of a representative experiment. (FIG. 3 panel C) Percentage of excision products relative to substrate was quantified based on three biological replicates. Data are means±SEMs; n=3. *P<0.05, **P<0.01, two-tailed unpaired t test.

FIG. 4 shows images of blots related to studies showing Decay kinetics of the EdU-induced excision products in human cells. (FIG. 4 panel A) In vivo excision assay showing EdU excision dynamics. HeLa cells were treated with 10 μM EdU, for 3, 6, 12, 24, and 48 h. Then, the excised oligonucleotides were isolated, immunoprecipitated with anti-TFIIH antibodies, mixed with a 50-mer internal control, 3′ end labeled, and separated on DNA-sequencing gels. (FIG. 4 panel B) In vivo excision assay of EdU pulse labeling and decay kinetics. HeLa cells were pulse labeled for 4 h in medium containing 10 μM EdU. Then, EdU-containing medium was removed and cells were supplied with fresh medium and incubated at 37° C. for 0, 3, 6, 9, and 12 h. Then, the excised oligonucleotides were isolated, immunoprecipitated with anti-TFIIH IP antibodies, and analyzed as in FIG. 4 panel A.

FIG. 5 shows images of data graphs related to studies showing XR-seq analysis which provides frequency distribution profiles of excision product length and nucleotide composition following different EdU treatment times. (FIG. 5 panel A) The size distribution of excision products peaks at 25 to 27 nt. (FIG. 5 panel B) Nucleotide distribution of the 26-mer and 27-mer excision products. Excision products generated by repair typically have the damaged bases located at positions 19 to 21; consistent with this is the peak in T residues (potential sites of EdU substitution) seen here surrounding position 20. Position is the distance in nucleotides from the 5′ end.

FIG. 6 shows images of data graphs related to studies showing EdU in the human genome is subject to transcription coupled repair. (FIG. 6 panel A) Browser view of representative genes show transcription-coupled repair. EdU repair on the HACD3, DHFR, and MSH3 genes. “+” indicates plus-strand DNA, “−” represents minus-strand DNA. Minus strand is the TS for HACD3 and MSH3, and the plus strand is the TS for DHFR. Individual bars represent the number of excision product reads as reads per kilobase per 10 million total reads on a scale from 0 to 7.5. Average repair of each gene was quantified in the bar chart in FIG. 6 panel B. (FIG. 6 panel C) Genome-wide EdU repair profile across all genes on the TS and NTS at 6, 9, 12, 24, and 48 h treatment times. In FIG. 6 panels B and C, RPKM is reads per kilobase per million total reads. Data for each strand were scaled to a unit gene to represent average repair in RNA Pol II-transcribed genes and the 2 kb upstream and downstream.

FIG. 7 shows images of a data graph and a blot related to studies showing absorbance of thymidine analogs. (FIG. 7 panel A) Thymidine analogs were diluted to 1 mM and absorbance of each was measured using a NanoDrop spectrophotometer. (FIG. 7 panel B) The nucleotide excision is not caused by EdU photoproducts but by EdU itself. HeLa cells were grown in a dark chamber with or without 10 μM EdU for 24 hours. Samples were either processed in the dark (protected from light) or under regular light conditions and analyzed on a sequencing gel.

FIG. 8 shows an image of a blot related to studies showing Taq DNA polymerase can bypass EdU adducts. Genomic DNA from Hela cells treated with or without 10 mM EdU for 24 hours was isolated. 50 ng of DNA was used as template for PCR amplification of the CSA gene (946bp) with Ta polymerase for 30 cycles. EdU substituted DNA template gave nearly as much full length PCR products as unsubstituted DNA.

FIG. 9 shows images of data graphs related to studies showing the effect of chromatin state on EdU repair. (FIG. 9 panel A) XR-seq read coverage was calculated over genomic intervals assigned to each of the chromatin states predicted for NHLF cells (ENCODE). Shown are results from EdU XR-seq at the different time points (6 hrs, 9 hrs, 12 hrs, 24 hrs, and 48 hrs). Values were normalized to read depth and interval length. (FIG. 9 panel B): Average EdU XR-seq profiles for each time point at 1.5-Kb regions flanking the center of DNase hypersensitivity peaks that either overlapped annotated genes (left) or did not overlap annotated genes (right).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Also, as used herein, “one or more” means one, two, three, four, five, six, seven, eight, nine, ten, 11, 12, 13, 14, 15, 16, 17, 18, 19, 20, etc.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

Amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage. As used herein, the term “amino acid” encompasses any naturally occurring amino acids, modified forms thereof, and synthetic amino acids, including non-naturally occurring amino acids. Alternatively, the amino acid can be a modified amino acid residue or can be an amino acid that is modified by post-translation modification (e.g., acetylation, amidation, formylation, hydroxylation, methylation, phosphorylation or sulfatation).

Unless the context indicates otherwise, it is specifically intended that the various features of the invention described herein can be used in any combination. Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

As used herein, the term “polypeptide” encompasses both peptides and proteins, unless indicated otherwise.

A “polynucleotide,” “nucleic acid,” or “nucleic acid molecule” as used herein is a sequence of nucleotide bases, and may be RNA, DNA or DNA-RNA hybrid sequences (including both naturally occurring and non-naturally occurring nucleotide), but in representative embodiments are either single or double stranded DNA sequences. As used herein, “nucleic acid molecule,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

A “therapeutic polypeptide” is a polypeptide or peptide that can alleviate, reduce, prevent, delay and/or stabilize symptoms that result from an absence or defect in a protein in a cell or subject and/or is a polypeptide that otherwise confers a benefit to a subject.

“Amino acid sequence” and terms such as “peptide,” “polypeptide,” and “protein” are used interchangeably herein, and are not meant to limit the amino acid sequence to the complete, native amino acid sequence (i.e., a sequence containing only those amino acids found in the protein as it occurs in nature) associated with the recited protein molecule. The proteins and protein fragments of the presently disclosed subject matter can be produced by recombinant approaches or can be isolated from a naturally occurring source. The protein fragments can be any size, and for example can range in size from four amino acid residues to the entire amino acid sequence minus one amino acid.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity. An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

A polypeptide, composition, compound or combination of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter).

As used herein, by “isolate” or “purify” (or grammatical equivalents) a vector, it is meant that the vector (e.g., a virus vector, e.g., an expression vector, e.g., a liposome, etc.) is at least partially separated from at least some of the other components in the starting material. In representative embodiments an “isolated” or “purified” vector is enriched by at least about 10-fold, 100-fold, 1000-fold, 10,000-fold or more as compared with the starting material.

As used herein, the term “modified,” as applied to a polynucleotide or polypeptide sequence, refers to a sequence that differs from a native/wildtype sequence due to one or more deletions, additions, substitutions, or any combination thereof.

A “heterologous nucleotide sequence” or “heterologous nucleic acid,” with respect to a cell, is a sequence or nucleic acid, respectively, that is not naturally occurring in the cell. Generally, the heterologous nucleic acid or nucleotide sequence comprises an open reading frame that encodes a polypeptide and/or a nontranslated RNA.

The term “fragment,” as applied to a peptide, will be understood to mean an amino acid sequence of reduced length relative to a reference peptide or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 4, 5, 6, 7, 8, 9, 10, 11, 12, 13, 14, 15 or more consecutive amino acids of a peptide or amino acid sequence according to the invention. In other embodiments, such fragments can comprise, consist essentially of, and/or consist of peptides having a length of less than about 15, 14, 13, 12, 11, 10, 9, 8, 7, 6, 5, 4, or less consecutive amino acids of a peptide or amino acid sequence according to the invention.

The “N-terminus” of a polypeptide is any portion of the polypeptide that starts from the N-terminal amino acid residue and continues to a maximum of the midpoint of the polypeptide.

The “C-terminus” of a polypeptide is any portion of the polypeptide that starts from the C-terminal amino acid residue and continues to a maximum of the midpoint of the polypeptide.

An isolated cell refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990). In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931). It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide can also function in intact cells without purification.

“Label” or “tag” as used herein may be any suitable label or detectable or otherwise functional group, e.g., a detectable moiety, including but not limited to biotin, avidin, fluorophores, antigens (including proteins and peptides), antibodies, porphyrins, radioactive or stable isotopes, etc., inclusive of examples as further described herein.

“Linker” or “linking group” as used herein may be any suitable linking group, including but not limited to groups comprising, consisting of or consisting essentially of C, O, N, P and/or S (e.g., including H where necessary). Linking groups that may be used to form covalent conjugates of two functional moieties are known in the art. See, e.g., U.S. Pat. Nos. 6,420,377; 6,593,334; and 6,624,317. The specific linking group employed will depend upon the particular synthetic method used to make the covalent conjugate, as will be appreciated by those skilled in the art. A suitable linking group will permit the joining of groups to provide a metabolically stable conjugate. In general, the linking moiety may comprise an aliphatic, aromatic, or mixed aliphatic and aromatic group (e.g., alkyl, aryl, alkylaryl, etc.) and contain one or more amino acids or hetero atoms such as N, O, S, etc.

The terms “antibody” and “immunoglobulin” include antibodies or immunoglobulins of any isotype, fragments of antibodies that retain specific binding to antigen, including but not limited to Fab, Fv, single chain Fv (scFv), Fc, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen (e.g., any antibody fragment that comprises at least one paratope).

Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include antibodies, antibody fragments, and nucleic acid sequences.

The term “contact” or grammatical variations thereof as used with respect to the interaction between two or more components, e.g., NSUN2 and an inhibitor thereof, e.g., NSUN2 and glucose, e.g., an inhibitor of NSUN2 and a cell, refers to bringing the two components in sufficiently close proximity to each other for one to exert a biological effect on the other. In some embodiments, the term contact means binding of the one component to the other.

The term “modulate,” “modulates,” or “modulation” refers to enhancement (e.g., an increase) or inhibition (e.g., a decrease) in the specified level or activity.

By “substantially retain” a property, it is meant that at least about 75%, 85%, 90%, 95%, 97%, 98%, 99% or 100% of the property (e.g., activity or other measurable characteristic) is retained.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40% , 45%, 50%, 55%, 60%, 65%, 70%, 75%, 80%, 85%, 90%, 95%, 97%, or 100%.

An “effective amount” or “therapeutically effective amount” refers to an amount of a compound or composition of this invention that is sufficient to produce a desired effect, which can be a therapeutic and/or beneficial effect. In general, a “therapeutically effective amount” or “treatment effective amount” refers to an amount that is a sufficient, but non-toxic, amount of the active ingredient (i.e., particles of this invention) to achieve the desired effect, which, for example, can be a reduction or elimination in the severity and/or frequency of symptoms and/or improvement or remediation of damage, or otherwise prevent, hinder, retard or reverse the progression of a disease or any other undesirable symptom. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)). The term “biologically active” as used herein means an enzyme or protein having structural, regulatory, or biochemical functions of a naturally occurring molecule. For example, as used herein, the term “therapeutically effective amount” or “effective amount” can refer to that amount of a pharmaceutical composition that results in amelioration of symptoms (e.g., reduction in size or elimination of a tumor) and/or a prolongation of survival in a subject. A therapeutically relevant effect relieves to some extent one or more symptoms of a disease or condition or returns to normal either partially or completely one or more physiological or biochemical parameters associated with or causative of the disease or condition.

As used herein, the terms “treating” or “treatment” of a condition or disease can include: (1) inhibiting the disease or condition, i.e., arresting, delaying or reducing the development of the disease or condition and its symptoms; or (2) relieving the disease or condition, i.e., causing regression of the disease or condition and its clinical symptoms. The term “treatment” or “treating,” as used herein, does not encompass 100% cure of cancer. However, in one embodiment, the therapeutic methods described herein can result in 100% reversal of detectable disease.

As used herein, the terms “prophylactic” or “preventative” treatment can include preventing at least one symptom of the disorder, disease or condition, i.e., causing a clinical symptom to not significantly develop in a subject that may develop or be predisposed to the disease but does not yet experience or display symptoms of the disease or condition.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. Preferably, the subject is a human. The subject may be of any gender, any ethnicity and any age. A “subject in need thereof” or “a subject in need of” is a subject known to have, or is suspected of having or developing or is at risk of having or developing disorder that can be treated by the methods and compositions of the present invention, or would benefit from the delivery of a particle and/or composition including those described herein.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compositions of the invention for a given set of conditions can be ascertained by those skilled in the art using conventional techniques and in view of the information set out herein. The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects. In some embodiments, more than one administration (e.g., two, three, four or more administrations) by one or more same and/or different routes of administration, may be employed to achieve the desired level of effect (e.g., a therapeutically effective amount) over a period of various intervals, e.g., daily, weekly, monthly, yearly, etc.

“Concurrently administering” or “concurrently administer” as used herein means that the two or more compounds or compositions are administered closely enough in time to produce a combined effect (that is, concurrently may be simultaneously, or it may be two or more events occurring within a short time period before and/or after each other, e.g., sequentially). Simultaneous concurrent administration may be carried out by mixing the compounds prior to administration, or by administering the compounds at the same point in time but at different anatomic sites and/or by using different routes of administration.

### Methods and Compositions

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

All compositions described herein may be useful in the methods described herein and/or other useful utility such as reagents for in vitro and/or in vivo research and development.

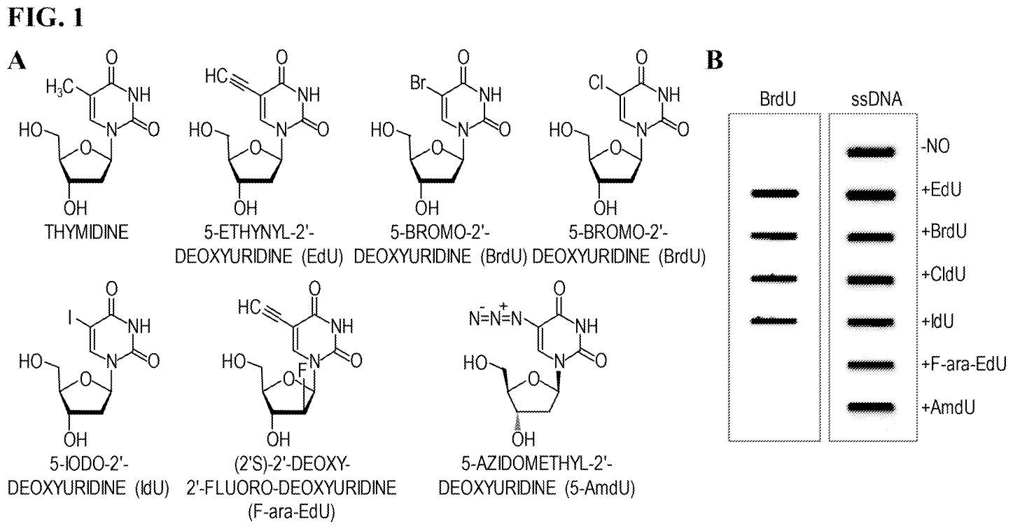

Thymidine isotopes (3H-thymidine, or 14C-thymidine, or 15N-thymidine) are modified nucleosides commonly used to label DNA in the 1950s because they are incorporated into DNA but not RNA (refs 1-4). The time-consuming and low resolution autoradiographic and density gradient separation assays used to detect incorporation of these isotopes in DNA led to the development of halogenated derivatives of thymidine as an alternative tool to study DNA synthesis. The chemical structures of these thymidine analogs are similar to thymidine (FIG. 1 panel A). In these analogues, the methyl group at the 5 position of the thymine ring is replaced by halogen atoms [bromine (BrdU), chlorine (CldU) or iodine (IdU)], and detection and visualization of these halogenated thymidine analogs is based on immunochemical and immunofluorescence detection with anti-BrdU antibodies which cross react with CldU and IdU. Without wishing to be bound to theory, the use of these analogues has traditionally required DNA denaturation with harsh chemical reagents to allow antibody accessibility of the halogenated thymidine derivatives in double stranded DNA, which leads to instability of DNA and disruption of other cellular components.

A strategy using 5-ethynyl-2′-deoxyuridine (EdU) and copper catalyzed azide alkyne cycloaddition (CuAAC) reaction (click chemistry) was developed to detect replicating DNA (ref 5). As used herein, “EdU” refers to the thymidine analogue 5-ethynyl-2′-deoxyuridine. EdU has a terminal alkyne group in the 5 position (FIG. 1 panel A) which can be coupled with other moieties such as, but not limited to, fluorescent- or biotin-labeled azides, allowing for fast, sensitive and high-throughput detection. EdU is now used in analysis of DNA replication (refs 5-9), cell proliferation and differentiation (ref 10), DNA combing (DNA fiber analysis), and measuring nucleotide excision repair synthesis in the form of “unscheduled DNA synthesis” (refs 11-14). EdU, like other thymidine analogs, also crosses the blood-brain-barrier and has been used to examine the limited DNA replication in brain cells (refs 5, 40, 41). However, studies of replication in the brain are limited because cell division in the brain is confined largely to the developmental phase. However, EdU is highly toxic (refs 15, 16), causing more cell death than the other thymidine analogs used to study replication, and the underlying molecular mechanism of toxicity is not known.

The present invention is based, in part, on the unexpected discovery that EdU is a substrate for excision repair when incorporated into the DNA of replicating cells. Without wishing to be bound to theory, the inventors of the present invention discovered that the thymidine analog EdU is processed as a “damage” in the human genome by the nucleotide excision repair system. Excision repair recognizes and removes bulky DNA lesions induced by a variety of DNA damaging agents, including environmental carcinogens such as UV (ref 17), benzo [a] pyrene (BaP) (ref 18) from cigarette smoke, and anti-cancer drugs such as cisplatin (refs 19, 20). Edu was found to be a substrate for excision repair, while the chemically related analogs BrdU, CldU, IdU, F-ara-EdU, or 5-AmdU were found not to be substrates for excision repair. It was observed that EdU was excised throughout the genome and was subject to transcription coupled repair as evidenced by higher repair rates in the transcribed strand (TS) relative to the nontranscribed strand (NTS) in transcriptionally active genes. Without wishing to be bound to theory, these high levels of excision repair in proliferative cells appeared to lead to cellular cytotoxicity by repeated EdU genome incorporation and excision therefrom. Without wishing to be bound to theory, these fatal cycles of high levels of excision repair appeared to continue at least in part due to the generation from the excision repair mechanisms of short nucleic acid molecules, oligomers comprising about 20 to about 30 nucleotides (e.g., about 20, 21, 22, 23, 24, 25, 26, 27, 28, 29, or 30 nucleotides), which would include the excised EdU, but none of the other thymidine analogs tested. These oligomers then degrade intracellularly, releasing free EdU to reincorporate into the genome of the proliferating cell, thereby initiating another cycle of EdU genome incorporation and excision therefrom leading to genomic DNA breaks. These properties of EdU, combined with its ability to cross the blood-brain barrier, make it a potential candidate for treatment of brain cancers, such as but not limited to glioblastomas.

Thus, one aspect of the present invention provides a method of treating a cancer located within the central nervous system in a subject in need thereof (e.g., wherein the subject has, is suspected to have, or is at risk of the cancer located within the central nervous system of the subject, e.g., a central nervous system cancer), comprising administering to the subject a therapeutically effective amount of 5-ethynyl-2′-deoxyuridine (EdU) or a nucleic acid molecule or composition comprising the same, thereby treating the cancer within the central nervous system of the subject.

Another aspect of the present invention provides a method of inhibiting and/or reducing growth of a cancer located within the central nervous system in a subject in need thereof (e.g., wherein the subject has, is suspected to have, or is at risk of the cancer located within the central nervous system of the subject, e.g., a central nervous system cancer), comprising administering to the subject a therapeutically effective amount of 5-ethynyl-2′-deoxyuridine (EdU) or a nucleic acid molecule or composition comprising the same, thereby inhibiting and/or reducing growth of the cancer in the subject.

Another aspect of the present invention provides a method of killing a cancer cell of a central nervous system cancer in a subject in need thereof (e.g., wherein the subject has, is suspected to have, or is at risk of the central nervous system cancer, e.g., a cancer located within the central nervous system of the subject), comprising: administering to the subject a therapeutically effective amount of 5-ethynyl-2′-deoxyuridine (EdU) or a nucleic acid molecule or composition comprising the same, wherein the EdU contacts the cancer cell within the central nervous system of the subject, thereby killing the cancer cell of the central nervous cancer in the subject.

Another aspect of the present invention provides a method of inhibiting and/or reducing proliferation of a cancer cell located within the central nervous system in a subject in need thereof (e.g., wherein the subject has, is suspected to have, or is at risk of cancer located within the central nervous system of the subject, e.g., a central nervous system cancer), comprising: administering to the subject a therapeutically effective amount of 5-ethynyl-2′-deoxyuridine (EdU) or a nucleic acid molecule or composition comprising the same, wherein the EdU contacts the cancer cell within the central nervous system of the subject, thereby inhibiting and/or reducing proliferation of the cancer cell in the subject.

In some embodiments, the EdU incorporates into the genome of a cancer cell located within the central nervous system of the subject.

In some embodiments, the genome-incorporated EdU is excised from the genome of the cancer cell located within the central nervous system of the subject.

In some embodiments, the genome-incorporated EdU is excised from the genome of the cancer cell located within the central nervous system of the subject and reincorporated into the genome of the cancer cell located within the central nervous system of the subject, optionally leading to the death (killing) of the cancer cell.

Cancers and cancer cells relevant in the present invention may be any type of cancer or cancer cell which may be located within the blood-brain barrier (BBB) of a subject, such as but not limited to a cancer or cancer cell of a spinal cancer and/or a brain cancer. Non-limiting examples of a spine cancer or brain cancer include glioblastoma [glioblastoma multiforme], oligodendroglioma, ependymoma, mixed glioma, choroid plexus tumors, ganglion cell tumors, embryonal tumors, meningioma, astrocytoma, lymphoma (e.g., primary CNS lymphoma), pituitary tumors, craniopharyngioma, germ cell tumors, non-meningothelial mesenchymal tumors, pineal region tumors, medulloblastomas, cancerous cysts, and/or metastatic tumors (e.g., cancers originating from other sources having metastasized to the central nervous system)).

In some embodiments, the brain cancer is glioblastoma multiforme, including but not limited to Proneural, Neural, Classical, and/or Mesenchymal glioblastoma subtype, such as described in cancergenome.nih.gov/researchhighlights/researchbriefs/foursubtypes.

In some embodiments of the methods of the present invention, the cancer cell may be in a subject, e.g., a mammalian subject, e.g., a human subject, e.g., a patient. In some embodiments, the cancer cell may be an in vitro cell, an ex vivo cell, or an in vivo cell.

In some embodiments, the subject is a mammal, e.g., a dog, a cat, a horse, a mouse, a rat, a non-human primate, a human. In particular embodiments, the subject is a human (e.g., a patient).

The EdU or nucleic acid molecule or composition comprising the same of the methods of the invention may be administered via any appropriate route, as would be within the knowledge of the skilled artisan, e.g., an oncologist, as needed for the conditions of the particular subject and/or patient. In some embodiments, the EdU or nucleic acid molecule or composition comprising the same may be administered topically (e.g., direct application), intravenously, cutaneously, subcutaneously, intraperitoneally, intra-arterially, intratumorally, intrathecally, intramuscularly, orally (e.g., by oral tablet or capsule), intranasally, sublingually, via inhalation, in an implant, in a matrix, in a gel, or any combination thereof.

Determination of a therapeutically effective and/or prophylactically effective amount, as well as other factors related to effective administration of a compound (e.g., EdU), nucleic acid molecule, and/or composition of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective and/or prophylactically effective treatment regimen for a subject of this invention is within the level of ordinary skill in the medical or veterinarian arts (e.g., an oncologist). In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

In some embodiments, the therapeutically effective amount of the EdU or a nucleic acid molecule or composition comprising the same may be about 1 mg/kg to about 1000 mg/kg or any value or range therein, e.g., about 1, 5, 10, 20, 30, 40, 50, 60, 70, 80, 90, 100, 150, 200, 250, 300, 350, 400, 450, 500, 550, 600, 650, 700, 750, 800, 850, 900, 950, or 1000 mg/kg or more. For example, in some embodiments, the therapeutically effective amount of the EdU or a nucleic acid molecule or composition comprising the same may be about 1 mg/kg to about 900 mg/kg, about 50 mg/kg to about 200 mg/kg, about 5 mg/kg to about 500 mg/kg, about 25 mg/kg to about 300 mg/kg, or about 50 mg/kg to about 1000 mg/kg, or any value or range therein. In some embodiments, the therapeutically effective amount of the EdU or a nucleic acid molecule or composition comprising the same may be about 1 mg/kg, 5 mg/kg, 50 mg/kg, 100 mg/kg, 200 mg/kg, 500 mg/kg, 750 mg/kg, or about 1000 mg/kg, or any value or range therein.

In some embodiments, the EdU or nucleic acid molecule or composition comprising the same may be delivered via one or more administration.

In some embodiments, the EdU or nucleic acid molecule or composition comprising the same may be delivered in via more than one administration, e.g., serial administrations.

In some embodiments, the one or more administrations may comprise administering the EdU or a nucleic acid molecule or composition comprising the same every 1, 2, 3, 4, 5, 6, 7, 8, 9, 10, 11, 12, 18, or 24 hrs, every 1, 2, 3, 4, 5, 6, 7, 14, 21, or 28 days, every 1, 2, 3, 4, 5, 6, or more weeks, every 1, 2, 3, 4, 5, 6, or more months, or any combination thereof (e.g., about 50 mg/kg/day for about 1 to about 15 days, e.g., about 200 mg/kg/day for about 1 to about 15 days, etc.).

In some embodiments, the methods of the present invention may further comprise the step of delivering one or more additional anti-cancer therapeutic agent or other cancer treatment.

In some embodiments, delivering an anti-cancer therapeutic agent or other cancer treatment may comprise delivering the anti-cancer therapeutic agent or other cancer treatment before, after, and/or concurrent with the delivery of the EdU or a nucleic acid molecule or composition comprising the same.

Non-limiting examples of an anti-cancer therapeutic agent or treatment include immunomodulatory agents, chemotherapy (e.g., cytotoxic) agents, anti-inflammatory agents, immunotherapy treatment (e.g., immunocheckpoint blockade agents), a surgical procedure, radiation, or any combination thereof. Additional non-limiting examples of anti-cancer therapeutic agents or other cancer treatments include surgery, radiation therapy, chemotherapy agents such as but not limited to daunomycin, cisplatin, oxaliplatin, carboplatin, verapamil, cytosine arabinoside, aminopterin, democolcine, tamoxifen, actinomycin D, alkylating agents (including, without limitation, nitrogen mustards, ethylenimine derivatives, alkyl sulfonates, nitrosoureas and triazenes), Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, Temozolomide, folic acid antagonists, pyrimidine analogs, purine analogs and adenosine deaminase inhibitors), Methotrexate, 5-fluorouracil (5-FU), Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Ara-C, paclitaxel, docetaxel, Mithramycin, Deoxyco-formycin, Mitomycin-C, L-Asparaginase, Interferons (e.g., IFN-α, IFN-β), interleukins, Etoposide, Teniposide, navelbene, CPT-11, anastrazole, letrazole, capecitabine, reloxafine, cyclophosphamide, ifosamide, droloxafine, melphalan, hexamethyl melamine, thiotepa, cytarabine, idatrexate, trimetrexate, dacarbazine, L-asparaginase, camptothecin, topotecan, bicalutamide, flutamide, leuprolide, pyridobenzoindole derivatives, PARP inhibitors (e.g., Olaparib), oligomycin, JQ1, emodin, metformin, shikonin, physcion (6PGD inhibitor), AICAR, oxythiamine, leflunomide, lonidamine, polydatin, honokiol, dehydropiandrosterone (DHEA), venetoclax (ABT-199, Bcl-2 inhibitor), navitoclax (ABT-263), A-1331852 (Bcl-xL inhibitor), ABT-737, S63845 (Mcl-1 inhibitor)); immunotherapy agents including but not limited to chimeric antigen receptor (CAR) cell therapy, monoclonal antibody therapy and/or immune checkpoint therapy (e.g., CAR-T therapy, CAR-NK therapy, anti-PD1, anti-PDL1, anti-CTLA4, anti-CD20, anti-EGFR, anti-VEGF, anti-VEGFR2, anti-TNFα, anti-CD44, anti-CD19, anti-CD3, anti-EpCAM, anti-IGF1R, anti-MUC1, anti-CD51, anti-integrin, or any other targeted antibody-based therapy with anti-cancer function), and any combination thereof.

In some embodiments, the EdU or nucleic acid molecule or composition comprising the same and the one or more additional anti-cancer therapeutic agent are administered as a single composition.

In some embodiments, the EdU or nucleic acid molecule or composition comprising the same and the one or more additional anti-cancer therapeutic agent are administered separately.

Also provided herein is a nucleic acid molecule (e.g., an isolated nucleic acid molecule), composition or combination comprising EdU.

In some embodiments, the nucleic acid molecule, composition or combination of the invention comprising EdU may be for the use of treating cancer and/or a cancer cell of a cancer located within the central nervous system.

In some embodiments, the nucleic acid molecule, composition or combination of the invention comprising EdU may be for the use of any of the methods disclosed herein.

In some embodiments, a nucleic acid molecule, combination or composition comprising EdU may be a non-naturally occurring molecule, combination or composition (e.g., an isolated, modified, synthetic, chimeric, recombinant and/or otherwise non-naturally nucleic acid molecule, combination or composition).

The EdU or nucleic acid molecule, combination or composition comprising the same of the invention can also be modified to comprise (e.g., linked, conjugated, bound (e.g., covalently bound, non-covalently bound), complexed, encapsulated, etc.) to and/or with one or more detectable moiety. Any suitable detectable moiety known in the art can be used in the practice of the present invention, and the detectable moiety can be detected using any method known in the art. For example, in some embodiments, the detectable moiety may be an exogenous epitope or chemical label that is attached to the nucleic acid molecule or composition comprising the EdU. Non-limiting examples of detectable moieties include an epitope, an enzyme, a ligand, a receptor, an antibody or antibody fragment and the like. In some embodiments, a detectable moiety of the invention may be a fluorescent moiety (e.g., GFP, EFP, RFP and the like), a peptide tag (e.g., FLAG, e.g., FLAG-tag), an antibody or fragment thereof, hemagglutinin antigen, polyHis, biotin, Protein A, streptavidin, maltose binding protein, c-myc, an enzyme such as glutathione-S-transferase, alkaline phosphatase, horseradish peroxidase, β-glucuronidase, β-galactosidase or luciferase, a radioactive moiety and/or an electron-dense moiety such as a ferritin or gold particle(s).

The detectable moiety can be detected either directly or indirectly using any suitable method. For example, for direct detection, the tag or reagent can comprise a radioisotope (e.g., ³⁵S) and the presence of the radioisotope detected by autoradiography. As another example, the tag or reagent can comprise a fluorescent moiety and be detected by fluorescence as is known in the art. Alternatively, the tag or reagent comprising the detectable moiety can be indirectly detected, i.e., the detectable moiety requires additional reagents to render it detectable. Illustrative methods of indirect labeling include those utilizing chemiluminescence agents, chromogenic agents, enzymes that produce visible reaction products, and ligands (e.g., haptens, antibodies or antigens) that may be detected by binding to labeled specific binding partners (e.g., hapten binding to a labeled antibody or a first antibody binding to a second antibody).

In some embodiments, the detectable moiety may be an antibody or antibody fragment. A variety of protocols for detecting the presence of and/or measuring the amount of antibodies or other polypeptides are known in the art. Examples of such protocols include, but are not limited to, enzyme-linked immunosorbent assays (ELISA), radioimmunoassays (RIA), radioreceptor assay (RRA), competitive binding assays and immunofluorescence microscopy. These and other assays are described, among other places, in Hampton et al. (Serological Methods, a Laboratory Manual, APS Press, St Paul, Minn (1990)) and Maddox et al. (J. Exp. Med. 158:1211-1216 (1993)).

In some embodiments, the EdU, nucleic acid molecule, composition or combination of the present invention may further comprise a pharmaceutically acceptable carrier (e.g., a pharmaceutical formulation). By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the EdU, nucleic acid molecule, composition or combination of the present invention may further comprise a pharmaceutical carrier, diluent, and/or adjuvant. In some embodiments, the EdU, nucleic acid molecule, composition or combination of the present invention may further comprise a pharmaceutical carrier, diluent, and/or adjuvant, and, optionally, other medicinal agents, therapeutic agents, pharmaceutical agents, stabilizing agents, buffers, carriers, adjuvants, diluents, etc., which can be included in the composition singly or in any combination and/or ratio.

Another aspect of the present invention relates to a kit comprising the EdU, nucleic acid molecule, composition or combination comprising the same, and useful for carrying out the methods of the invention. The kit may further comprise additional reagents for carry out the methods (e.g., buffers, containers, additional therapeutic agents) as well as instructions for use.

Some kits include EdU and/or nucleic acid molecules and/or compositions of the invention in a container (e.g., vial or ampule), and may also include instructions for use of the EdU and/or nucleic acid molecules and/or compositions of the invention in the various methods disclosed above. EdU and/or nucleic acid molecules and/or compositions of the invention can be in various forms, including, for instance, as part of a solution or as a solid (e.g., lyophilized powder). The instructions may include a description of how to prepare (e.g., dissolve or resuspend) the EdU and/or nucleic acid molecules and/or compositions of the invention in an appropriate fluid and/or how to administer the EdU and/or nucleic acid molecules and/or compositions of the invention for the treatment of the diseases and disorders described herein.

The kits may also include various other components, such as buffers, salts, complexing metal ions and other agents described above in the section on pharmaceutical compositions. These components may be included with the EdU and/or nucleic acid molecules and/or compositions of the invention or may be in separate containers. The kits may also include other therapeutic agents for administration with the EdU and/or nucleic acid molecules and/or compositions of the invention. Examples of such agents include, but are not limited to, agents to treat the disorders or conditions described above.

Another aspect of the invention provides for preparation of a medicament for use comprising the EdU, nucleic acid molecule, composition or combination of the present invention.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1:

Incorporation of thymidine analogs into the human genome and removal of 5-ethynyl-2′-deoxyuridine by nucleotide excision repair: UV-irradiated human cells excise cyclobutane pyrimidine dimers (CPDs) and (6-4) photoproducts [(6-4) PPs] in the form of nominal 26-mer nucleotide-long oligomers (refs 24-26) which may be purified by damage-or TFIIH-specific antibodies to be sequenced and mapped to the genome by the XR-seq method (refs 17, 19, 20, 26, 27). In addition to using damage-specific or TFIIH-specific antibodies to capture the excised oligomers, this study employed antibodies to readily available pyrimidine analogs (FIG. 1 panel A). EdU, BrdU, CldU, and IdU can all be immunoprecipitated with anti-BrdU antibodies (refs 28, 29) and are all similarly incorporated into replicating HeLa cells (FIG. 1 panel B).

Before employing the anti-BrdU antibodies to capture the excised oligomers from UV-irradiated cells, a series of experiments were performed as intended negative control of UV treatment. HeLa cells replicating in the presence of either EdU, BrdU, CldU, or IdU were collected, lysed, and supernatants were immunoprecipitated with anti-BrdU antibodies. The eluted material was mixed with an internal standard control 50-mer, radiolabeled, and separated on a sequencing gel. Surprisingly, the BrdU antibodies precipitated oligomers in the form of nominal 26-mer from EdU-labeled cells, but not cells labeled with BrdU, CldU, or IdU (FIG. 1 panel C). These results raised the possibility that EdU in the genome was recognized as damage and processed by nucleotide excision repair as such.

Repair deficient cells do not excise EdU, and other thymidine analogs are not excision repair substrates: To confirm that EdU containing DNA is a bona fide nucleotide excision repair substrate, repair functionality was assayed using an XPA-/-XPC-/-CSB-/- derivative of NHF1 cells which lack the capacity to perform either global or transcription-coupled repair. FIG. 1 panel D shows that the mutant has a clean background and no excision product in contrast to the excision product seen with the wild type NHF-1 parental cell line. Collectively, these data suggest that EdU is removed from the genome by nucleotide excision repair.

Further to the finding that EdU is a substrate for the human excision nuclease, screens were performed of the other two thymidine analogs (F-ara-EdU and AmdU) that are used to monitor cell proliferation and DNA replication dynamics but not recognized by anti-BrdU antibodies by isolating the potential excision products containing the analogs using TFIIH antibodies. It had been previously shown that excised oligonucleotides remain in a tight complex with TFIIH which also protect them from non-specific nucleolytic degradation following release from the genomic DNA (refs 17, 30, 31). FIG. 1 panel E shows that with anti-TFIIH antibodies, only EdU gives rise to the about 25-32 nucleotide-long excision products and as noted, under these conditions, only the undegraded primary excision product is detected, in contrast to the BrdU IPs where the smaller degraded excision products are also seen (FIG. 1 panels C and D). Collectively, these data indicate that of all the thymidine analogs currently in use for studying DNA replication, only EdU is excised by human nucleotide excision repair.

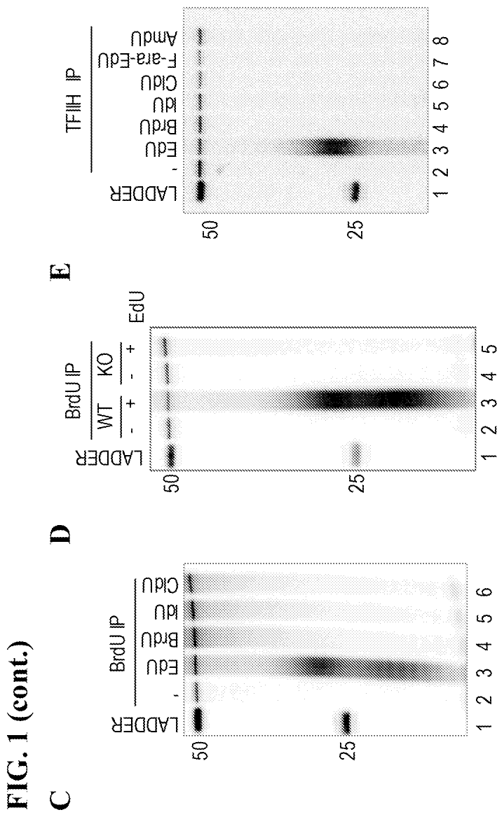

Capturing excised oligomers from UV-irradiated cells with anti-BrdUJ antibodies: An initial goal of the study was to employ anti-BrdU antibodies to capture the excised oligomers from UV-irradiated cells. HeLa cells were therefore incubated in medium containing either BrdU or EdU for 24 h before UV irradiation, to permit incorporation of the analogs into the genome, and thus, the excision products. Then, 1h after UV irradiation (20 J/m2, 254 nm) cells were collected, lysed, small oligonucleotides were immunoprecipitated, mixed with an internal standard control 50 mer, radiolabeled, and separated on a sequencing gel. UV excision products were detected in cells incubated with either BrdU or with EdU, but not in the cells without analog (FIG. 2). A similar amount of EdU-containing oligos are excised in the presence and absence of UV, which indicated that EdU is a good substrate for nucleotide excision repair.

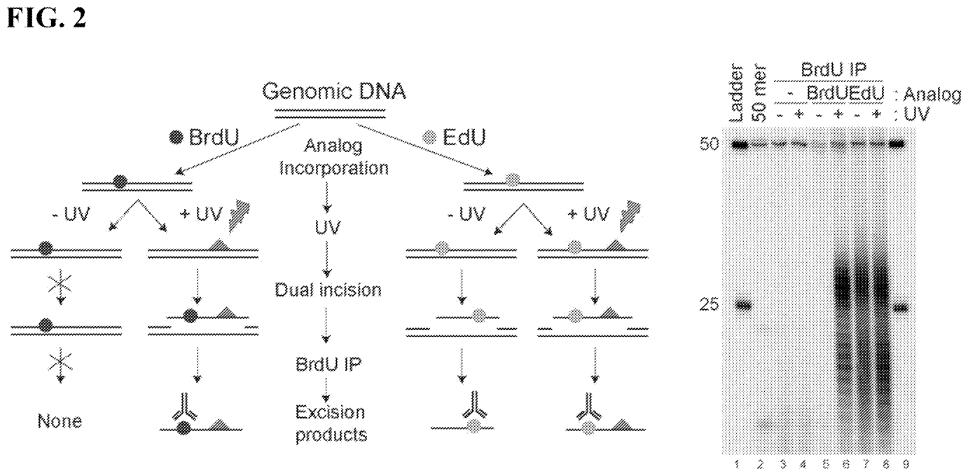

Repair of 5-ethynyl-2′-deoxyuridine by mammalian cell free extract: To further confirm that EdU is a bona fide substrate for excision repair and to compare its excision relative to a (6-4) photoproduct, in vitro assays were conducted with mammalian cell-free extracts using duplex DNA substrates (FIG. 3 panel A) that were either unmodified (UM), possessing one or two EdU residues, or a (6-4) photoproduct adjacent to a ³²P radiolabel (ref 35). As shown in FIG. 3 panel B, both EdU substrates were excised with products in the form of a nominal 26 mer(22-30 nt in length) comparable to the (6-4) photoproduct. Quantification of the percentage of excision relative to total amount of substrate based from three biological replicates (FIG. 3 panel C) showed that the substrate containing 1 EdU was excised less efficiently compared with the (6-4) PP. The substrate with 2 EdUs gave a stronger signal than the substrate with 1 EdU, which indicates that 2 EdUs may cause more severe DNA helical distortions than 1 EdU. Thus, the in vitro excision assay confirmed that EdU is an efficient substrate for the mammalian excision nucleases.

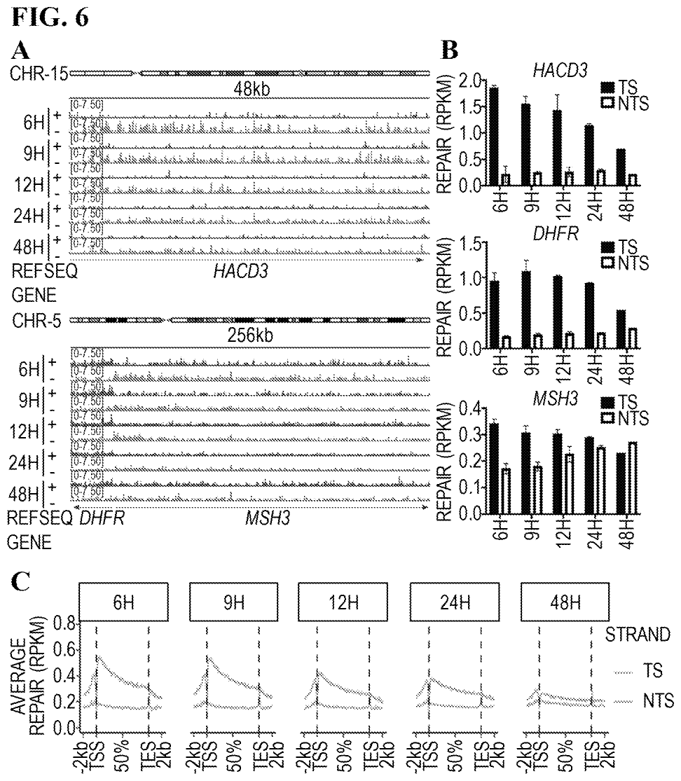

Light-independent EdU excision: EdU has a high extinction coefficient in the 280-300 nm region (FIG. 7 panel A), it was possible that cells labeled with EdU exposed to the light in the room during routine manipulation might have formed an EdU adduct with another cellular component generating a “bulky” adduct recognizable by the excision nuclease. To address that possibility, a carefully controlled experiment was conducted in which one set of the assays were performed under yellow light and one set under ordinary room light (FIG. 7 panel B). Under both conditions, cells grown in EdU-containing media excised oligonucleotides 22-30 nts in length. Collectively, these data indicate that the excision is not caused by an EdU photoproduct but by EdU itself.

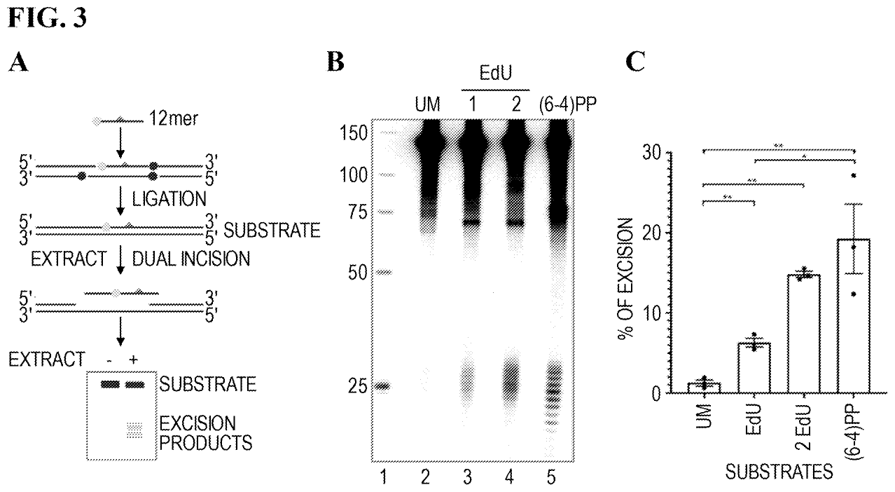

DNA-EdU substrate formation and decay kinetics: To determine the optimal time for EdU substrate formation in the genome, HeLa cells were incubated in medium containing 10 μM EdU for 3 to 48 hrs, whereupon the cells were lysed, excision products were isolated using BrdU antibodies and the excised EdU-containing fragments detected by 3′ labeling and autoradiography. FIG. 4 panel A shows that the peak level is reached at 24 h and declines by 48 h. However, the 48 h decline is due to the loss of total genomic DNA caused by cell death, potentially the consequence of the long EdU exposure and continuous excision and re-synthesis. To determine the kinetics of excision after initial incorporation of EdU, cells were incubated with EdU for 4 hours, then the medium replaced with EdU-free medium and EdU excision examined at timepoints up to 12 hrs. As seen in FIG. 4 panel B, removal continues for at least 12 hrs, albeit with a gradual decrease in the excision products.

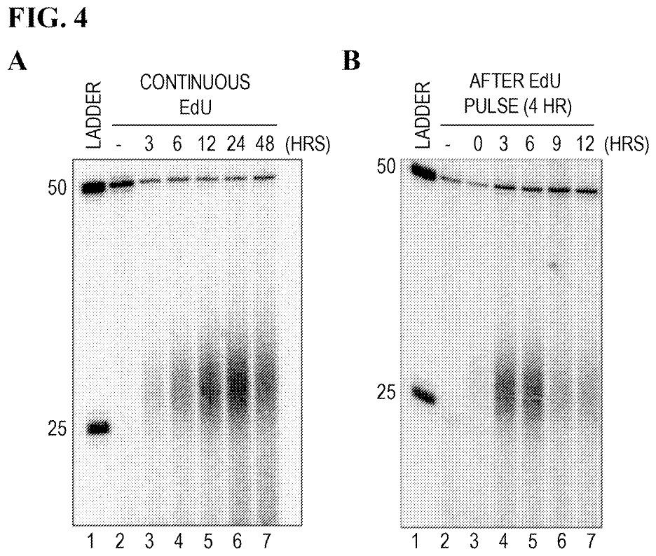

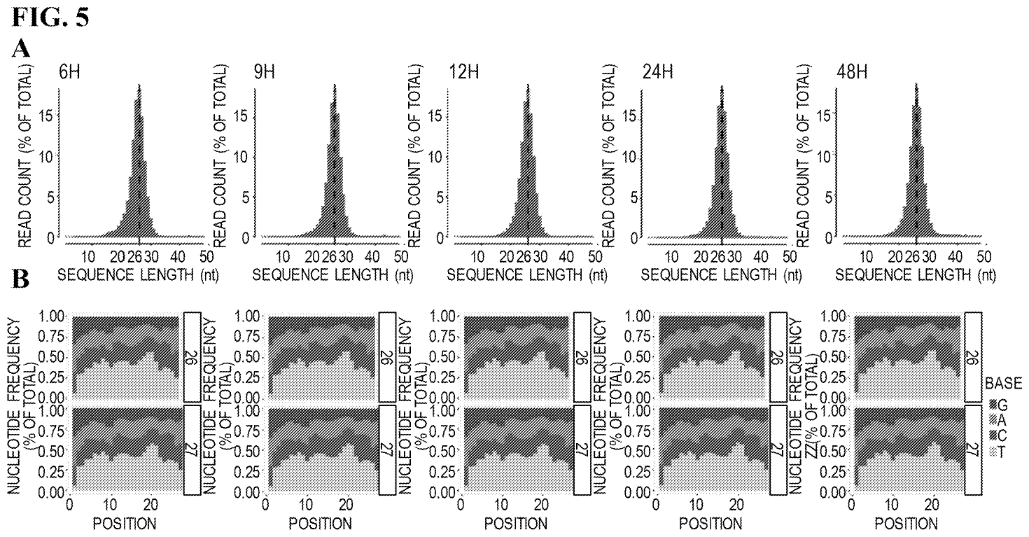

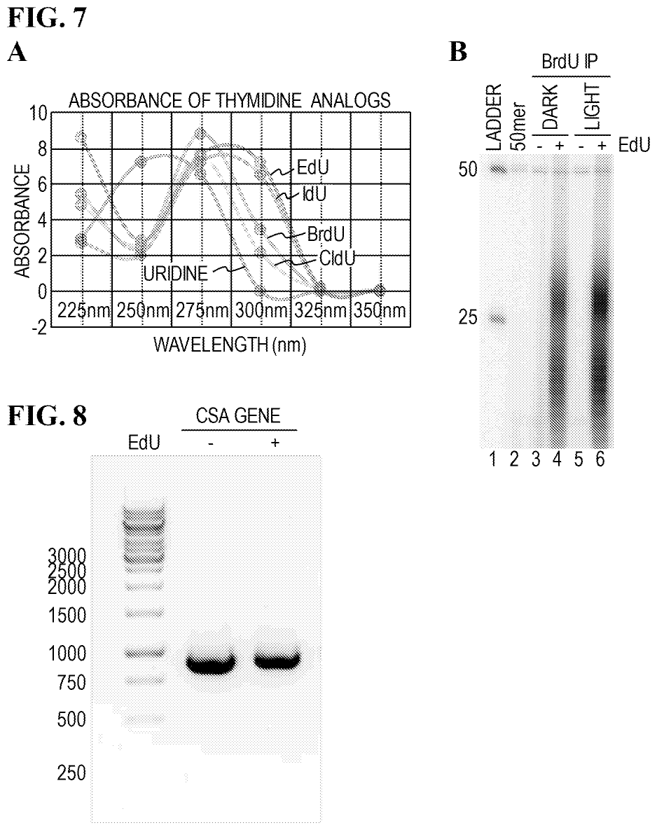

EdU in the human genome is subject to transcription coupled repair: Structurally, the ethynyl group at the C-5 position in Uridine in EdU is not very different from the-CH3 group at this position of thymidine. Hence it was not clear whether EdU would arrest DNA or RNA polymerases which in the latter case is assumed to be important for DNA adduct removal by transcription-coupled repair (TCR). EdU as a potential polymerase block was tested by performing PCR using Taq DNA polymerase and genomic DNAs from untreated cells or cells treated with EdU for 24 hrs. It was found that under both conditions the PCR reaction produced full-size fragments (FIG. 8). To determine whether EdU would block RNA Pol II, and thus be subjected to TCR or not, studies were performed using XR-seq on excision products immunoprecipitated with anti-TFIIH antibodies and thus presumed to be the primary reaction products. FIG. 5 panel A shows the size distribution of the excision products which exhibited the expected narrow length distribution with a peak at 25-27 nucleotides. The nucleotide distribution of the excision products in this range showed enrichment of Ts at positions 19 and 20 for 26-mers and at positions 20-21 for the 27-mer (FIG. 5 panel B). Browser views of the HACD3, DHFR, and MSH3 genes which have been extensively used for studying TCR of UV damage (ref 17) clearly showed TCR (FIG. 6 panel A). The TS/NTS repair ratio peaks at 6 h and gradually decreased from 9 h to 48 h (FIG. 6 panel B).

The plots in FIG. 6 panel C show analysis of TS vs NTS repair genome-wide. XR-seq reads from non-overlapping genes known to be transcribed by RNA Pol II were scaled to a unit gene, and reads 2 kb upstream and downstream of each gene were included in the plots. The TS/NTS ratio is ˜2.5 at 6 h and went down to ˜1.2 at 48 h, with higher values in promoter-proximal than distal regions, and there is a switch of preferential repair upstream of the transcription start site (TSS) known to be caused by anti-sense transcription in the promoter and enhancer regions of RNA Pol II transcribed genes (ref 27).

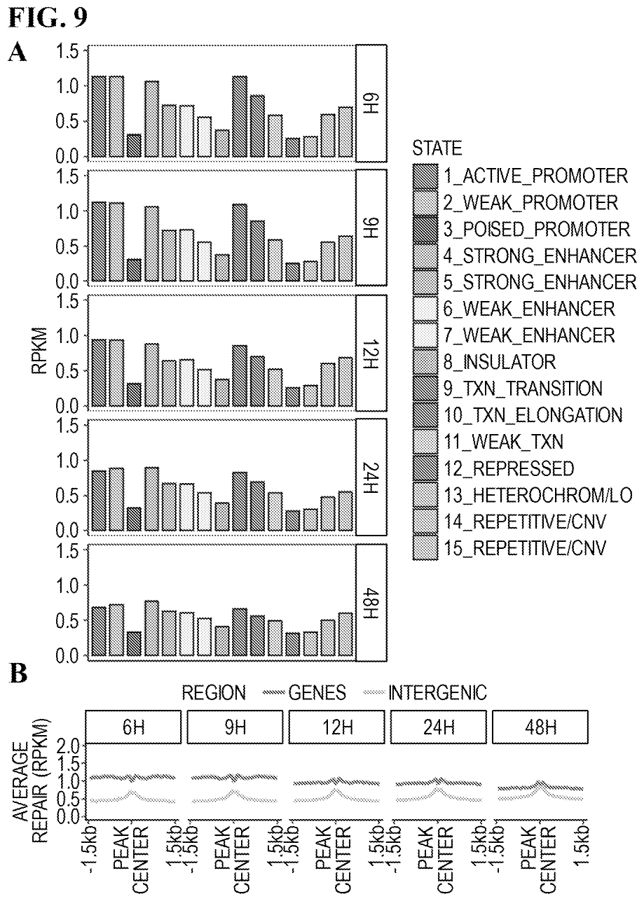

Effect of chromatin state on EdU repair: Open or closed chromatin affects the accessibility of repair proteins and further affects repair efficiency. 15 chromatin states were predicted by the ChromHMM algorithm based on chromatin modifications (ref 36). Previous reports have showed that chromatin states affect the repair efficiency of CPD, (6-4)PP (ref 37), cisplatin (ref r38) and BPDE-dG adducts (ref 18). To test if the same also applied to EdU in the genome, in this study the number of XR-seq reads in each chromatin state were calculated, and it was found that the reads were relatively high over the active promoter, weak promoter, strong enhancer and transcription transition region (FIG. 9 panel A), and these reads decreased over time (6 h to 48 h). This enrichment of reads at active chromatin regions is additional evidence that EdU repair is transcription-coupled. Next, the EdU XR-seq reads were mapped over the DNase hypersensitivity peaks of gene and intergenic regions. Repair of EdU was significantly enriched at all DNA hypersensitivity peaks and the repair at gene regions was more efficient than intergenic regions because it is more accessible by repair proteins, and repair of EdU in gene regions gradually went down with time (FIG. 9 panel B). Repair of EdU around the DNase hypersensitivity peaks of intergenic regions went up with time as the repair of gene regions decreased.

Cancers are characteristically composed of dividing cells. EdU has been shown to outperform BrdU in suppressing cell proliferation of an osteosarcoma and a glioblastoma cell line (ref 21). However, even though EdU had similar effect in suppressing primary human glioblastoma cell proliferation as Temozolomide (standard of care in treating glioblastoma), it induced 3-5-fold more strand breaks and apoptosis than Temozolomide at the same dose (ref 21). The cytotoxic properties of EdU as described herein, combined with its ability to cross the blood-brain barrier, make it a candidate for treatment of brain cancers.

### Example 2: Materials and Methods as Used in Example 1.

Cell lines: NHF1 cells, telomerase-immortalized normal human fibroblast monolayers were obtained as described previously (ref 17), XPA-/-XPC-/-CSB-/- triple knock out NHF1 cells were generated using CRISPR Cas9 system with gRNAs (XPA: GGCGGCTTTAGAGCAACCCG (SEQ ID NO:1); CSB: CGTGGAGAAGGAGTATCGGT (SEQ ID NO:2); XPC: CGAGATGTGGACACCTACTA (SEQ ID NO:3)). The HeLa cell line was from the American Type Culture Collection (ATCC). NHF1 and HeLa cells were maintained in Dulbecco's modified Eagle's medium (DMEM) supplemented with 10% fetal bovine serum at 37° C. in a 5% CO2 humidified chamber.

Reagents and antibodies: Thymidine analogs 5-Ethynyl-2′-deoxyuridine (EdU:900584), 5-Bromo-2′-deoxyuridine (BrdU:B5002), 5-Iodo-2′-deoxyuridine (IdU:17125), 5-Chloro-2′-deoxyuridine (CldU:C6891), (2′S)-2-Deoxy-2-fluoro-5-ethynyluridine (F-ara-EdU: T511293), and 5-(azidomethyl)-2′-deoxyuridine (AmdU:T342254) were purchased from Sigma. Anti-BrdU antibody (Bu20A) was from Thermo (14-5071-82); Anti-p89 (sc-293), and anti-p62 (sc-292) were purchased from Santa Cruz Biotechnology; Anti-ssDNA antibody was from Millipore Sigma (MAB3034). Undamaged (AGGAATTAAGGA; SEQ ID NO:4), single EdU (AGGAATEdUAAGGA; SEQ ID NO:5) and double EdU (AGGAAEdUEdUAAGGA: SEQ ID NO:6) modified oligomers were synthesized by Integrated DNA Technologies (IDT).

UV Irradiation and Thymidine analog treatment: HeLa cells were grown to ˜80% confluence, culture medium was removed, cells were washed twice with PBS, and then were placed under a GE germicidal lamp emitting primarily 254-nm UV light (1 J/m²/sec) connected with a digital timer for 20sec (20 J/m² in total). Cells were collected after 1 hour repair. Thymidine analogs were dissolved in sterile PBS (BrdU, EdU, CldU) or DMSO (IdU, F-ara-EdU, AmdU), and then added to cell culture medium at final concentration of 10 μM for indicated times.

Slot blot assay: HeLa cells were plated in 100 mm plates and treated with 10 μM thymidine analogs for 24 h. Genomic DNA was purified from cell pellets using QIAamp DNA mini kit (Qiagen, 51306). DNA concentrations were determined using a Qubit 3.0 fluorometer. 250 ng of each DNA was diluted with TE buffer (10 mM Tris-HCl pH8, 1 mM EDTA) to a total volume of 250 μL, then boiled at 90° C. for 10 min and cooled in ice water immediately. 250 μL cold ammonium acetate (2 M) was added to neutralize DNA, and then DNA samples were loaded onto nitrocellulose membranes using a slot blot apparatus. Membranes were transferred to a vacuum oven and baked for 1.5 h at 80° C., then blocked with 5% dried milk and incubated with either anti-BrdU antibody (1:1000) or anti-ssDNA antibody (1:5000) as loading control overnight at 4° C. After incubating with a horseradish peroxidase (HRP) labeled secondary antibody (1:10000) for 1 h, chemiluminescent signal was captured using a Bio-Rad ChemiDoc XRS imaging device.

In vivo excision assay: For in vivo excision assay with BrdU antibody, low molecular DNAs isolated by Hirt procedure as previously described (ref 17) were subject to immunoprecipitation with anti-BrdU antibody. For in vivo excision assay with TFIIH antibody, cells were lysed in buffer A (25 mM HEPES at pH7.9, 100 mM KCl, 12 mM MgCl2, 0.5 mM EDTA, 2 mM DTT, 12.5% glycerol, 0.5% NP-40), and excised oligonucleotides complexed with TFIIH protein were immunoprecipitated with TFIIH antibody. Purified oligonucleotides from BrdU IP or TFIIH IP were 3′ end-labeled by terminal deoxynucleotidyl transferase (TdT), α-³²P-ATP with a standard 50 mer oligonucleotide as spike internal control. After phenol-chloroform extraction and ethanol precipitation, labeled DNAs were fractionated with 12% denaturing sequencing gels.

In vitro excision assay: Radiolabeled 140-bp full length undamaged, (6-4) PP, and EdU damaged substrates were prepared, and in vitro excision assay with cell-free extract from Chinese hamster ovary (CHO) AA8 cells were carried out as previously described (ref 39). Briefly, Chinese hamster ovary (CHO) AA8 cell free extract (CFE) was prepared by the method of Manley (ref 42). Linear double-stranded DNA substrates (140bp in length) were prepared with centrally located (6-4) photoproduct or EdU as described previously (ref 43). The sequence of the centrally located 12-mers was 5′-AGGAATTAAGGA; SEQ ID NO:4. For the (6-4) substrate, the (6-4) lesion was between T5 and T6. Single EdU was at T6 and, double EdU lesions were at T5 and T6. Unmodified 12mer and EdU containing 12mers were purchased from Integrated DNA Technologies.

The excision reaction with AA8 CFE (75 μg) was conducted with 20 fmol of 140bp substrates, 18 mM HEPES-KOH (pH 7.9), 24 mM KCl, 2 mM MgCl2, 4 mM ATP at 30° C. for 60 minutes as described (ref 35). After the excision reaction, the mixture was incubated with 0.34% SDS and 20 g/ml Proteinase K at 55-60° C. for 15 minutes, then DNA was extracted with phenol:chloroform:isoamyl alcohol and precipitated with ethanol, resuspended in formamide/dye mixture, and separated with a 10% sequencing gel. Quantification of the signal intensities was done by using ImageJ.

XR-seq: The XR-seq experiment was done as previously reported (refs 17, 27) with modifications. In brief, HeLa cells were treated with 10 μM EdU and harvested after 6 h, 12 h and 24 h. Cell pellets from two 150 mm culture plates were suspended and lysed in 1 mL cold buffer A (25 mM HEPES at pH7.9, 100 mM KCl, 12 mM MgCl2, 0.5 mM EDTA, 2 mM DTT, 12.5% glycerol, 0.5% NP-40). The primary EdU excision products were isolated by immunoprecipitation with TFIIH antibody followed by ligation of 5′ and 3′ adapters. After adaptor ligation, excised oligomers containing EdU were further purified by immunoprecipitation with anti-BrdU antibody that also reacts with EdU. Purified EdU excision oligomers were directly subjected to PCR amplification using 50- and 63-nt-long primers that introduce specific barcodes compatible with the Illumina TruSeq small RNA kit, without a damage reversal step as is done with CPD or (6-4) PP photoproducts. The PCR products containing excised oligonucleotides were ˜145 base pairs (bp) in length and resolved with a 10% nondenaturing gel. EdU excision oligomers from different time points were gel purified, pooled and sequenced on a NextSeq-P3 platform.

Data analysis: Analysis of sequencing reads and data visualization were as described previously (ref 19). Reads were trimmed to remove flanking adapter sequences by cutadapt (ref 44), and then duplicate reads were removed by fastx_toolkit/0.0.14 (hannonlab.cshl.edu/fastx_toolkit/index.html). Trimmed reads were aligned to hg38_UCSC by using bowtie2 with arguments-f-very-sensitive (refs 45, 46). The output sam files were converted into bam files by using samtools (ref 47) and then were converted into bed files using bedtools (ref 48). Oligonucleotide lengths and nucleotide distributions were plotted by R. Only the reads of 26-mer length with T at 19 and 27-mer length with T at 20 were analyzed.

Bigwig files were visualized by IGV (RRID: SCR_007073, Broad Institute, and the Regents of the University of California) (refs 49, 50). RPKM for each gene was plotted with Prism 9 (RRID: SCR_002798). For plotting average repair profiles as a unit gene, we chose the genes with length >5kbp, and the distance between genes was at least 5kbp. For chromatin state analysis, bedtools coverage was used to calculate the repair levels over each of the 15 predicted chromatin states defined by the ChromHMM algorithm (ref 36). Values were normalized per million mapped reads and per Kb of interval length and plotted with R (github.com/yanyanyangunc/DNA-Damage-Repair-Circadian-Clock/tree/master/excisionRepair). DNase-seq (Accession No. ENCSR000EMP) fastq, aligned reads.bam files, and peak files, as well as the NHLF chromHMMchromatin state segmentation (UCSC Accession No. wgEncodeEH000792), were downloaded from the ENCODE portal (genome.ucsc.edu/ENCODE/). The raw data and alignment data have been deposited in the Gene Expression Omnibus under accession numbers GSE202784.

### Example 3: EdU in Tumor Xenograft Models

Male and female athymic mice (nu/nu genotype, Balb/c background, 6 to 8 weeks old) will be used for all antitumor studies. The animals will be maintained in Thoren ventilated cage and rack system (Allentown, PA). All animal procedures will conform to the appropriate Institutional Animal Care and Use Committee and NIH guidelines.

Tumor xenografts and implantation: For intracranial (i.c.) studies, s.c. xenografts passaged in athymic mice will be excised from the host mice under sterile conditions in a laminar flow containment hood. The xenograft will be minced and the cells separated with a 60-mesh tissue cytosieve (BioWhittaker Inc., Walkersville, MD) into a ZO solution (Sigma Aldrich, Allentown, PA), allowing for passage through a 25-gauge needle. After centrifugation, the supernatant will be removed, and the cells will be mixed 1:1 with methylcellulose. This mixture will then loaded into a repeating 250-/J Hamilton syringe (Hamilton, Co., Reno, NV) dispenser and injected i.c. at an inoculation volume of 10 μL. The i.c. injections will be performed by placing a mouse into a stereotactic frame. A ½″ midline skin incision will be made. The bregma is located and the coordinates (2 mm lateral) will be determined. A mounting holder on the frame supports the syringe containing the cells. A sterile 25-gauge needle attached to the syringe will be introduced through the calvaria and into the brain at a depth of 4 mm. The needle will then pulled back 0.5 mm to create a “well” for the homogenate. The xenograft homogenate will be injected and after 1 minute the syringe will be pulled up and a small amount of bone wax will be placed to occlude the hole. The mouse will then be removed from the frame and surgical glue will used to close the skin (Carlson et al, 2011).

Intracranial xenograft therapy: For i.c. tumor studies, groups of mice will be randomized at a time point that represents ½ of its median day to Test Out (TO). For example, if an untreated IC PDX line would test out at day 30, treatment (Rx) would start at day 15, 40 TO/20 Rx, 50 TO/25 Rx etc. days after i.c. tumor implantation.

Treatment Groups will be about 10 animals per group (8 for efficacy study & 2 for XR-Seq analysis) Three groups will include: (1) Thymidine Control 50 mg/kg/day IP×15 days: (2) EdU 50 mg/kg/day IP×15 days; and (3) EdU 200 mg/kg/day IP×15 days. Each group will be treated for 5-7 days and brain tissue will be harvested 4 hours post injection and stored on ice.

Evaluation of intracranial xenograft response: The response of the i.c. xenografts to treatment will be assessed by the percentage of increase in time to a specific neurologic endpoint (i.e., seizure activity, repetitive circling, 15% decrease in weight or decrease in appetite) or to moribund status. Statistical analysis will be performed using the Wilcoxon rank order test. All animals will be observed twice daily for signs of distress or development of neurological symptoms, at which time they will be removed from the study.

### References

1 P. Reichard, B. Estborn, Journal of Biological Chemistry 188, 839-846 (1951).
2. M. Downing, B. Schweigert, Journal of Biological Chemistry 220, 521-526 (1956).
3. M. Friedkin, D. Tilson, D. Roberts, Journal of Biological Chemistry 220, 627-637 (1956).
4. J. H. Taylor, P. S. Woods, W. L. Hughes, Proceedings of the National Academy of Sciences of the United States of America 43, 122 (1957).
5. A. Salic, T. J. Mitchison, Proceedings of the National Academy of Sciences 105, 2415-2420 (2008).
6. T. Yokochi, D. M. Current Protocols in Cell Biology 35, 22.10. 21-22.10. 14 (2007).
7. A. Ligasová, K. Koberna, Molecules 23, 3007 (2018).
8. G. M. Solius, D. I. Maltsev, V. V. Belousov, O. V. Podgorny, Journal of Biological Chemistry 297 (2021).
9. A. Ligasová et al., Open biology 6, 150172 (2016).
10. O. Cinquin, S. L. Crittenden, D. E. Morgan, J. Kimble, Proceedings of the National Academy of Sciences 107, 2048-2053 (2010).
11. S. Limsirichaikul et al. Nucleic acids research 37, e31-e31 (2009).
12. G. C. Stefos, E. Szantai, D. Konstantopoulos, M. Samiotaki, M. Fousteri, Nucleic acids research 49, e64-e64 (2021).
13. W. Wu et al., Nature 593, 440-444 (2021).
14. D. A. Reid et al., Science 372, 91-94 (2021).
15. P. J. Barr, P. A. Nolan, D. V. Santi, M. J. Robins, Journal of medicinal chemistry 24, 1385-1388 (1981).
16. R. Walker et al. (1980) The synthesis and properties of some 5-substituted uracil derivatives. in Nucleic Acids Symposium Series, pp s95-102.
17. J. Hu, S. Adar, C. P. Selby, J. D. Lieb, A. Sancar, Genes & development 29, 948-960 (2015).
18. W. Li et al., Proceedings of the National Academy of Sciences 114, 6752-6757 (2017).
19. Y. Yang et al., Proceedings of the National Academy of Sciences 115, E4777-E4785 (2018).
20. A. Yimit, O. Adebali, A. Sancar, Y. Jiang, Nature communications 10, 1-11 (2019).
21. H. H. Ross et al., Journal of neuro-oncology 105, 485-498 (2011).
22. E. I. Fomchenko, E. C. Holland, Clinical Cancer Research 12, 5288-5297 (2006).
23. P. C. Huszthy et al., Neuro-oncology 14, 979-993 (2012).
24. R. Boyce, R. Setlow, The action spectra for ultraviolet-light inactivation of systems containing 5-bromouracil-substituted deoxyribonucleic acid: I. Escherichia coli 15 T-A-U-. Biochimica et Biophysica Acta (BBA)-Specialized Section on Nucleic Acids and Related Subjects 68, 446-454 (1963).
25. R. D. Wood, Journal of Biological Chemistry 272, 23465-23468 (1997).
26. A. Sancar, Mechanisms of DNA repair by photolyase and excision nuclease (Nobel Lecture). Angewandte Chemie International Edition 55, 8502-8527 (2016).
27. J. Hu et al., Nature protocols 14, 248-282 (2019).
28. H. G. Gratzner, Science 218, 474-475 (1982).
29. R. Liboska, A. Ligasová, D. Strunin, I. Rosenberg, K. Koberna, PloS one 7, e51679 (2012).
30. M. G. Kemp, J. T. Reardon, L. A. Lindsey-Boltz, A. Sancar, Journal of Biological Chemistry 287, 22889-22899 (2012).
31. J. Hu et al., Journal of Biological Chemistry 288, 20918-20926 (2013).
32. R. Setlow, R. Boyce, Biochimica et Biophysica Acta (BBA)-Specialized Section on Nucleic Acids and Related Subjects 68, 455-461 (1963).
33. J. D. Regan, R. Setlow, R. Ley, Proceedings of the National Academy of Sciences 68, 708-712 (1971).
34. B. L. Cavanagh, T. Walker, A. Norazit, A. C. Meedeniya, Molecules 16, 7980-7993 (2011).
35. M. E. Branum, J. T. Reardon, A. Sancar, Journal of Biological Chemistry 276, 25421-25426 (2001).
36. J. Ernst et al., Nature 473, 43-49 (2011).
37. S. Adar, J. Hu, J. D. Lieb, A. Sancar, Proceedings of the National Academy of Sciences 113, E2124-E2133 (2016).
38. J. Hu, J. D. Lieb, A. Sancar, S. Adar, Proceedings of the National Academy of Sciences 113, 11507-11512 (2016).
39. J. T. Reardon, A. Sancar, Methods in enzymology 408, 189-213 (2006).
40. F. Chehrehasa, A. C. Meedeniya, P. Dwyer, G. Abrahamsen, A. Mackay-Sim, Journal of neuroscience methods 177, 122-130 (2009).
41. C. Zeng et al., Brain research 1319, 21-32 (2010).
42. J. L. Manley, A. Fire, A. Cano, P. A. Sharp, M. L. Gefter, Proceedings of the National Academy of Sciences 77, 3855-3859 (1980).
43. J.-C. Huang, A. Sancar, Journal of Biological Chemistry 269, 19034-19040 (1994).
44. M. Martin, EMBnet. journal 17, 10-12 (2011).
45. B. Langmead, S. L. Salzberg, Nature methods 9, 357-359 (2012).
46. B. Langmead, C. Wilks, V. Antonescu, R. Charles, Bioinformatics 35, 421-432 (2019).
47. H. Li et al., Bioinformatics 25, 2078-2079 (2009).
48. A. R. Quinlan, Current protocols in bioinformatics 47, 11.12. 11-11.12. 34 (2014).
49. J. T. Robinson et al., Nature biotechnology 29, 24-26 (2011).
50. H. Thorvaldsdottir, J. T. Robinson, J. P. Mesirov, Briefings in bioinformatics 14, 178-192 (2013)
51. Carlson B L, Pokorny J L, Schroeder M A, Sarkaria J N. Curr Protoc Pharmacol. 2011; 52 (14):1-14.
52. Keir S T, Hausheer F, Lawless A A, Bigner D D, Friedman H S. Cancer Chemother Pharmocol. 2001; 48(1):83-7.
53. Keir S T, Dewhirst M W, Kirkpatrick J P, Bigner D D, Batinic-Haberle I. Anticancer Agents Med Chem. 2011; 11(2):202-12.

The foregoing is illustrative of the present invention, and is not to be construed as limiting thereof. The invention is defined by the following claims, with equivalents of the claims to be included therein.

1. A method of treating a cancer located within the central nervous system in a subject in need thereof, comprising administering to the subject a therapeutically effective amount of 5-ethynyl-2′-deoxyuridine (EdU) or a nucleic acid molecule or composition comprising the same, thereby treating the cancer within the central nervous system of the subject.

2. A method of inhibiting and/or reducing growth of a cancer located within the central nervous system in a subject in need thereof , comprising administering to the subject a therapeutically effective amount of 5-ethynyl-2′-deoxyuridine (EdU) or a nucleic acid molecule or composition comprising the same, thereby inhibiting and/or reducing growth of the cancer in the subject.

3. A method of killing a cancer cell of a central nervous system cancer in a subject in need thereof , comprising:
administering to the subject a therapeutically effective amount of 5-ethynyl-2′-deoxyuridine (EdU) or a nucleic acid molecule or composition comprising the same, wherein the EdU contacts the cancer cell within the central nervous system of the subject,
thereby killing the cancer cell of the central nervous cancer in the subject.

4. (canceled)

5. The method of claim 1, wherein the EdU incorporates into the genome of a cancer cell located within the central nervous system of the subject.

6. The method of claim 5, wherein the genome-incorporated EdU is excised from and optionally reincorporated into the genome of the cancer cell located within the central nervous system of the subject.

7. The method of claim 1, wherein the cancer is a spinal cancer and/or a brain cancer.

8. The method of claim 7, wherein the brain cancer is glioblastoma multiforme.

9. The method of claim 1, wherein the subject is a mammal.

10. The method of claim 1, wherein the subject is a human.

11. The method of any one of claim 1, wherein the EdU or composition comprising the same is administered to the subject topically, intravenously, cutaneously, subcutaneously, intraperitoneally, intra-arterially, intratumorally, intrathecally, intramuscularly, orally, intranasally, sublingually, via inhalation, in an implant, in a matrix, in a gel, or any combination thereof.

12. The method of claim 1, wherein the therapeutically effective amount of the EdU or a nucleic acid molecule or composition comprising the same is about 1 mg/kg to about 1000 mg/kg.

13. The method of claim 1, wherein the EdU or composition comprising the same is delivered via two or more administrations.

14. (canceled)

15. The method of claim 1, further comprising delivering one or more additional anti-cancer therapeutic agent or other cancer treatment.

16. The method of claim 15, wherein the anti-cancer therapeutic agent or other cancer treatment is surgery, radiation therapy, chemotherapy (e.g., daunomycin, cisplatin, oxaliplatin, carboplatin, verapamil, cytosine arabinoside, aminopterin, democolcine, tamoxifen, actinomycin D, alkylating agents (including, without limitation, nitrogen mustards, ethylenimine derivatives, alkyl sulfonates, nitrosoureas and triazenes), Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, Temozolomide, folic acid antagonists, pyrimidine analogs, purine analogs and adenosine deaminase inhibitors), Methotrexate, 5-fluorouracil (5-FU), Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Ara-C, paclitaxel, docetaxel, Mithramycin, Deoxyco-formycin, Mitomycin-C, L-Asparaginase, Interferons (e.g., IFN-α, IFN-β), interleukins, Etoposide, Teniposide, navelbene, CPT-11, anastrazole, letrazole, capecitabine, reloxafine, cyclophosphamide, ifosamide, droloxafine, melphalan, hexamethyl melamine, thiotepa, cytarabine, idatrexate, trimetrexate, dacarbazine, L-asparaginase, camptothecin, topotecan, bicalutamide, flutamide, leuprolide, pyridobenzoindole derivatives, PARP inhibitors (e.g., Olaparib), oligomycin, JQ1, emodin, metformin, shikonin, physcion (6PGD inhibitor), AICAR, oxythiamine, leflunomide, lonidamine, polydatin, honokiol, dehydropiandrosterone (DHEA), venetoclax (ABT-199, Bcl-2 inhibitor), navitoclax (ABT-263), A-1331852 (Bcl-xL inhibitor), ABT-737, S63845 (Mcl-1 inhibitor)), immunotherapy (e.g., chimeric antigen receptor (CAR) cell therapy, monoclonal antibody therapy and/or immune checkpoint therapy (e.g., CAR-T therapy, CAR-NK therapy, anti-PD1, anti-PDL1, anti-CTLA4, anti-CD20, anti-EGFR, anti-VEGF, anti-VEGFR2, anti-TNFα, anti-CD44, anti-CD19, anti-CD3, anti-EpCAM, anti-IGF1R, anti-MUC1, anti-CD51, anti-integrin, or any other targeted antibody-based therapy with anti-cancer function), and any combination thereof.

17. The method of claim 15, wherein the EdU or composition comprising the same and the one or more additional anti-cancer therapeutic agent are administered as a single composition.

18. The method of claim 15, wherein the EdU or composition comprising the same and the one or more additional anti-cancer therapeutic are administered separately.

19. An isolated nucleic acid molecule comprising EdU for the use in the method of claim 1.

20. A composition comprising EdU or a nucleic acid molecule comprising the same for the use in the method of claim 1.

21. (canceled)

22. A kit comprising the composition of claim 20, and optional instructions for the use thereof.

23. The method of claim 7, wherein the spinal cancer and/or a brain cancer is glioblastoma [glioblastoma multiforme], oligodendroglioma, ependymoma, mixed glioma, choroid plexus tumors, ganglion cell tumors, embryonal tumors, meningioma, astrocytoma, lymphoma, primary CNS lymphoma, a pituitary tumor, craniopharyngioma, a germ cell tumor, a non-meningothelial mesenchymal tumor, a pineal region tumor, medulloblastoma, a cancerous cyst, and/or a metastatic tumor originating from other sources having metastasized to the central nervous system.
